(19) United States
(12) Patent Application Publication
(10) Pub. No.: US 2025/0322951 A1
(43) Pub. Date: Oct. 16, 2025

(54) SYSTEM AND METHOD FOR VALIDATING A CLASSIFICATION CODE ASSIGNED TO A DATA OBJECT BY A FIRST ARTIFICIAL INTELLIGENCE (AI) MODEL USING A SECOND AI MODEL

(71) Applicant: Optum, Inc., Minnetonka, MN (US)

(72) Inventor: Amit KUMAR, Gaya (IN)

(21) Appl. No.: 18/633,751

(22) Filed: Apr. 12, 2024

(51) Int. Cl. G16H 50/20; G16H 10/60

(52) U.S. Cl. CPC G16H 50/20; G16H 10/60

(57) ABSTRACT

Systems and methods for validating a classification code assigned to a data object by a first artificial intelligence (AI) model using a second AI model are provided. A data object associated with an entity including a classification code that is assigned to the data object via the first AI model can be received. The classification code for the data object that is assigned to the data object via the first AI model can be validated using the second AI model. The second AI model can be trained using positive data objects that include assigned classification codes that are correct and negative data objects that include assigned classification codes that are incorrect. A validation result can be transmitted to a user device based on validating the classification code for the data object using the second AI model.

### TECHNICAL FIELD

The present disclosure relates, generally, to the technical field of data validation and AI. More specifically, the present disclosure relates to validating a classification code that is assigned to a data object by a first AI model using a second AI model.

### BACKGROUND

An AI model can be trained to assign a classification code to a data object. A major challenge in building any sort of predictive AI model that can associate the relation between data objects and classification codes is that the underlying classification codes might not have any semantic structure. As such, generating a semantic representation of a classification code might not be feasible. Accordingly, the accuracy and quality of AI models that assign classification codes to data objects might be reduced.

### SUMMARY

It can be understood that both the foregoing general description and the following detailed description are explanatory only and are not restrictive of the disclosure, as claimed.

According to an aspect, a computer-implemented method can include receiving, by one or more processors, a data object associated with an entity including a classification code that is assigned to the data object via a first artificial intelligence (AI) model; validating, by the one or more processors and using a second AI model, the classification code for the data object that is assigned to the data object via the first AI model, wherein the second AI model is trained using positive data objects that include assigned classification codes that are correct and negative data objects that include assigned classification codes that are incorrect; and transmitting, by the one or more processors and to a user device, a validation result based on validating the classification code for the data object using the second AI model.

According to another aspect, a device can include a memory configured to store instructions; and one or more processors configured to execute the instructions to perform operations comprising: receiving, by the one or more processors, a data object associated with an entity including a classification code that is assigned to the data object via a first artificial intelligence (AI) model; validating, by the one or more processors and using a second AI model, the classification code for the data object that is assigned to the data object via the first AI model, wherein the second AI model is trained using positive data objects that include assigned classification codes that are correct and negative data objects that include assigned classification codes that are incorrect; and transmitting, by the one or more processors and to a user device, a validation result based on validating the classification code for the data object using the second AI model.

According to another aspect, a non-transitory computer-readable medium can store instructions that, when executed by one or more processors, cause the one or more processors to perform operations comprising: receiving, by one or more processors, a data object associated with an entity including a classification code that is assigned to the data object via a first artificial intelligence (AI) model; validating, by the one or more processors and using a second AI model, the classification code for the data object that is assigned to the data object via the first AI model, wherein the second AI model is trained using positive data objects that include assigned classification codes that are correct and negative data objects that include assigned classification codes that are incorrect; and transmitting, by the one or more processors and to a user device, a validation result based on validating the classification code for the data object using the second AI model.

### BRIEF DESCRIPTION OF THE DRAWINGS

FIG. 1 is a diagram of an example system for validating a classification code that is assigned to a data object.

FIG. 2 is a diagram of example components of one or more devices of FIG. 1.

FIG. 3 is a diagram of an AI model for validating a classification code that is assigned to a data object.

FIG. 4 is a flowchart of an example process for training an AI model for validating a classification code that is assigned to a data object.

FIG. 5 is a diagram of an example process for training an AI model for validating a classification code that is assigned to a data object.

FIG. 6 is a diagram of an example process for training a contrastive loss function.

FIG. 7 is a flowchart of an example process for validating a classification code that is assigned to a data object using an AI model.

### DETAILED DESCRIPTION

The present disclosure relates, generally, to the technical field of data validation and AI. More specifically, the present disclosure relates to validating a classification code that is assigned to a data object by a first AI model using a second AI model.

Conventional techniques for validating coding of classification codes to data objects can be tedious, subjective, time consuming, error-prone, and expensive. In some cases, an AI model can assign a classification code to a data object. However, the AI model might inaccurately assign the classification code. Also, major challenges in building any sort of predictive AI model that can associate the relation between data objects and classification codes is that classification codes might not have any semantic structure. As such, generating a semantic representation of a classification codes might not be feasible.

Some embodiments of the present disclosure provide a system and method for validating a classification code assigned to a data object by a first AI model using a second AI model. For instance, some embodiments learn the sematic representation of the classification code along with data objects so that using both of the representations provides a predictive AI model that can validate whether an assigned classification code by an AI model is correct or is incorrect. In this way, some embodiments of the present disclosure provide an improvement to the accuracy and quality of classification coding.

The above technical improvements, and additional technical improvements, will be described in detail throughout the present disclosure. Also, it should be apparent to a person of ordinary skill in the art that the technical improvements of the embodiments provided by the present disclosure are not limited to those explicitly discussed herein, and that additional technical improvements exist.

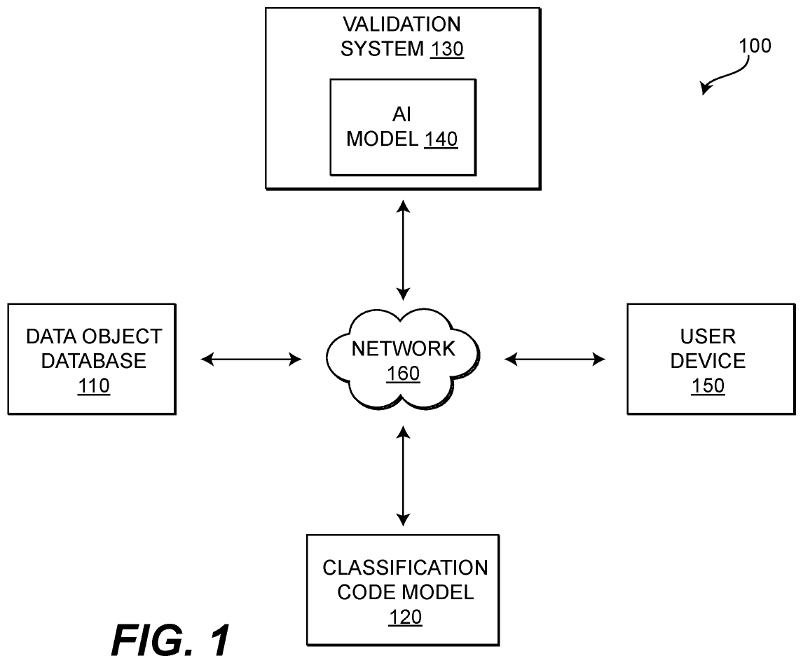

FIG. 1 is a diagram of an example system 100 for validating a classification code that is assigned to a data object. Throughout this disclosure, a classification code includes a diagnostic code such as, e.g., a hierarchical condition category code (HCC), and a data object includes a data object associated with an entity such as, e.g., a medical record associated with a patient. As shown in FIG. 1, the system 100 can include a data object database 110, a classification code model 120, a validation system 130, an AI model 140, a user device 150, and a network 160.

The example system 100 provides a deep-learning based semi-supervised framework for validating and improving the precision of pre-assigned classification codes to data objects.

The data object database 110 can be configured to store data objects of entities. For example, the data object database 110 can be a cloud database, a hierarchical database, a network database, a relational database, or the like. The data objects of the entities can be medical records such as electronic health records (EHRs), electronic data objects (EMRs), personal health records (PHRs), clinical charts, or the like. The data objects can include text describing diagnoses of entities, medical conditions of entities, drugs prescribed to entities, operations performed on entities, or the like.

The classification code model 120 can be configured to assign a classification code to a data object. For example, the classification code model 120 can be an AI model such as a neural network, a linear regression model, a decision tree model, a supper vector machine, or the like. The classification code can be an HCC code, an International Classification of Diseases (ICD) code, or the like.

The validation system 130 can be configured to validate, using the AI model 140, a classification code assigned to a data object by the classification code model 120. For example, the validation system 130 can be a server, a desktop computer, a smartphone, laptop computer, or the like.

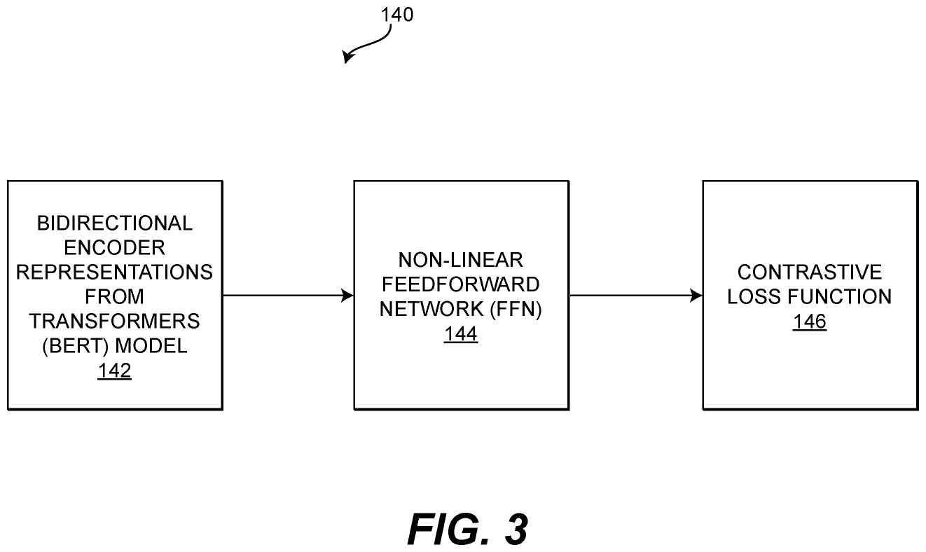

The AI model 140 can be configured to validate a classification code assigned to a data object by the classification code model 120. For example, the AI model 140 can be a language model, a neural network, or the like. As a particular example, and as described in relation to FIG. 3, the AI model 140 can include a bidirectional encoder representations from transformers (BERT) model 142, a non-linear feed-forward network (FFN) 144, and a contrastive loss function 146. According to another embodiment, the AI model 140 can include a transformer-based encoder. According to another embodiment, the AI model 140 can include joint attention modules.

The user device 150 can be configured to display a validation result indicating whether a classification code assigned to a data object is correct. For example, the user device 150 can be a desktop computer, a laptop computer, a smartphone, a wearable device, or the like.

The network 160 can be configured to permit communication between the devices of FIG. 1. For example, the network 160 can be a cellular network (e.g., a fifth generation (5G) network, a long-term evolution (LTE) network, a third generation (3G) network, a code division multiple access (CDMA) network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, or the like, and/or a combination of these or other types of networks.

The number and arrangement of the devices of the system 100 shown in FIG. 1 are provided as an example. In practice, the system 100 can include additional devices, fewer devices, different devices, or differently arranged devices than those shown in FIG. 1. Additionally, or alternatively, a set of devices (e.g., one or more devices) of the system 100 can perform one or more functions described as being performed by another set of devices of the system 100.

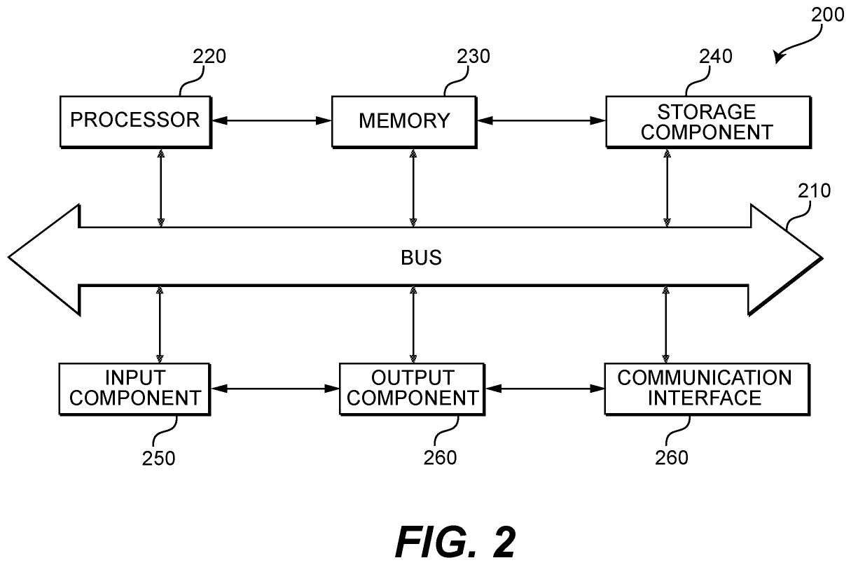

FIG. 2 is a diagram of example components of a device 200 of FIG. 1. The device 200 can correspond to the data object database 110, the validation system 130, and/or the user device 150.

As shown in FIG. 2, the device 200 can include a bus 210, a processor 220, a memory 230, a storage component 240, an input component 250, an output component 260, and a communication interface 270.

The bus 210 includes a component that permits communication among the components of the device 200. The processor 220 can be implemented in hardware, firmware, or a combination of hardware and software. The processor 220 can be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 220 can include one or more processors capable of being programmed to perform a function. The memory 230 can include a random access memory (RAM), a read only memory (ROM), and/or another type of dynamic or static storage device (e.g., a flash memory, a magnetic memory, and/or an optical memory) that stores information and/or instructions for use by the processor 220.

The storage component 240 can store information and/or software related to the operation and use of the device 200. For example, the storage component 240 can include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 250 can include a component that permits the device 200 to receive information, such as via user input (e.g., a touch screen display, a keyboard, a keypad, a mouse, a button, a switch, and/or a microphone for receiving the reference sound input). Additionally, or alternatively, the input component 250 can include a sensor for sensing information (e.g., a global positioning system (GPS) component, an accelerometer, a gyroscope, and/or an actuator). The output component 260 can include a component that provides output information from the device 200 (e.g., a display, a speaker for outputting sound at the output sound level, and/or one or more light-emitting diodes (LEDs)).

The communication interface 270 can include a transceiver-like component (e.g., a transceiver and/or a separate receiver and transmitter) that enables the device 200 to communicate with other devices, such as via a wired connection, a wireless connection, or a combination of wired and wireless connections. The communication interface 270 can permit the device 200 to receive information from another device and/or provide information to another device. For example, the communication interface 270 can include an Ethernet interface, an optical interface, a coaxial interface, an infrared interface, a radio frequency (RF) interface, a universal serial bus (USB) interface, a Wi-Fi interface, a cellular network interface, or the like.

The device 200 can perform one or more processes described herein. The device 200 can perform these processes based on the processor 220 executing software instructions stored by a non-transitory computer-readable medium, such as the memory 230 and/or the storage component 240. A computer-readable medium can be defined herein as a non-transitory memory device. A memory device can include memory space within a single physical storage device or memory space spread across multiple physical storage devices.

The software instructions can be read into the memory 230 and/or the storage component 240 from another computer-readable medium or from another device via the communication interface 270. When executed, the software instructions stored in the memory 230 and/or the storage component 240 can cause the processor 220 to perform one or more processes described herein. Additionally, or alternatively, hardwired circuitry can be used in place of or in combination with software instructions to perform one or more processes described herein. Thus, implementations described herein are not limited to any specific combination of hardware circuitry and software.

The number and arrangement of the components shown in FIG. 2 are provided as an example. In practice, the device 200 can include additional components, fewer components, different components, or differently arranged components than those shown in FIG. 2. Additionally, or alternatively, a set of components (e.g., one or more components) of the device 200 can perform one or more functions described as being performed by another set of components of the device 200

FIG. 3 is a diagram of an AI model 140 for validating a classification code that is assigned to a data object. As shown in FIG. 3, the AI model 140 can include a BERT model 142, a FFN 144, and a contrastive loss function 146.

The BERT model 142 can be configured to generate a representation vector from a data object. The BERT model 142 can be represented as:

$${u}_{i}^{p} = f ( {\hat{x}}_{i}^{p} ) , where f ( ) is the BERT Model 142$$

### Here,

$${u}_{i}^{p}$$

can be the encoder representation on an i-th data object of entity p and

$${u}_{i}^{d}$$

### ∈R^{d}.

The representation vector can be represented as:

$${v}_{i}^{p} = g ( {h}_{i}^{d} )$$

Here, g( ) can be the embedding layer that initializes the hidden representation classification code of a positive data object.

The validation system 130 can use a large volume of corpus which is unlabeled from another domain by using transfer learning. The BERT model 142 can be built as an extension of a base model, which can incorporate the cross-domain transfer. As different domains come with domain-specific regularities, the text representation learned on one domain can not produce optimal performance on another domain. The validation system 130 can learn a neural model that transfers knowledge from a source domain to a target domain for the task of classification code assignment. The codes used as labels that are used in the source domain can also available for assignment in the target domain. The validation system 130 can use the base model to learn a preliminary embedding of data sets in the source domain and the target domain. The validation system 130 can re-train the base BERT model 142, which is originally pre-trained on clinical notes from a database using a method of masked language modeling on the two tasks of masked token prediction and next sentence prediction.

The validation system 130 can use the pre-trained BERT model 142 to extract representation vectors from positive data objects and negative data objects. Although the BERT model 142 is described in connection with various embodiments of the present disclosure, the validation system 130 can use any other suitable framework as an alternative to the BERT model 142.

The FFN 144 can be configured to map the semantic representation (e.g., representation vector) and a classification code to a space where contrastive loss is applied. The FFN 144 can be represented as:

$${y}_{i}^{p} = m ( {v}_{i}^{p} ) = σ ( {v}_{i}^{p} + {w}^{2} ( {v}_{i}^{p} ) ) where σ is a RELU non linearity$$

### Here,

$${z}_{i}^{p} and {y}_{i}^{p}$$

can be the projected representation vectors of data objects and classification codes for an i-th visit of entity p.

The contrastive loss function 146 can be configured to learn and identify a positive data object and representation vector of the positive data object, and learn a randomly initialized classification code representation. Given a set

$$\{ {\hat{x}}_{i}^{p} , {\hat{x}}_{2}^{p} , … … , {\hat{x}}_{i}^{p} \}$$

that includes positive data objects

$${\hat{x}}_{i}^{p}$$

ana negative data objects

$${\hat{x}}_{j}^{p}$$

for a entity p, the contrastive loss function 146 can be configured to differentiate the existence of classification code

$${h}_{i}^{p}$$

in the positive data objects

$${\hat{x}}_{i}^{p}$$

using contrastive learning loss.

The contrastive loss function 146 can be represented as:

$${ℒ}^{k} = - log ( exp ( sim ( {u}_{i}^{p} , {y}_{i}^{p} ) / τ ) / {\sum }_{j = 0}^{N} exp ( sim ( {u}_{j}^{p} , {y}_{i}^{p} ) ) )$$

Here, [symbol] ^{k} can be the contrastive loss function of k-th training example of entity p,

$${u}_{i}^{p} , {u}_{j}^{p}$$

are projected representations of positive data objects and negative data objects.

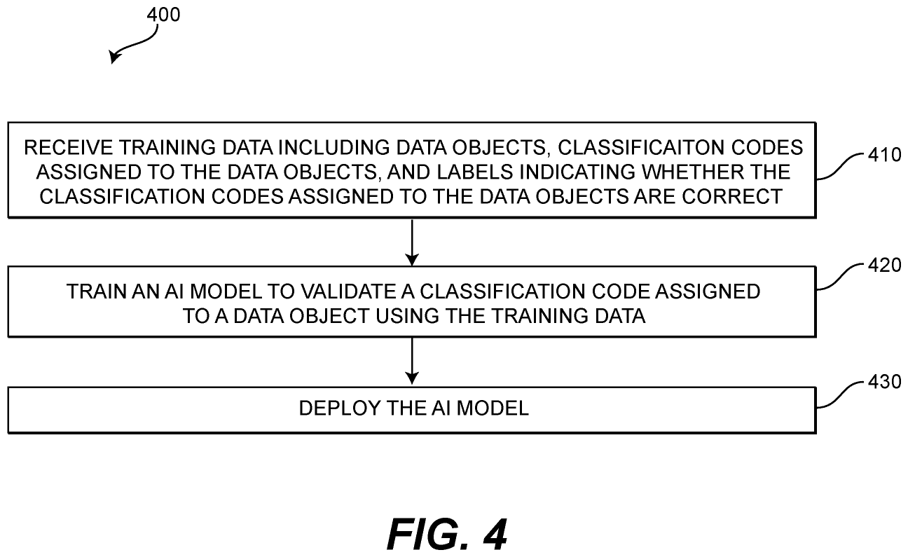

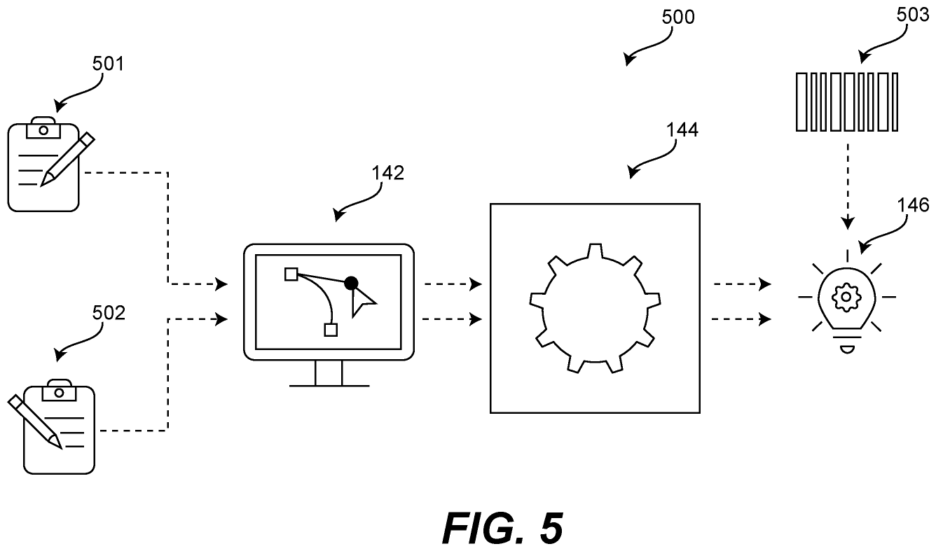

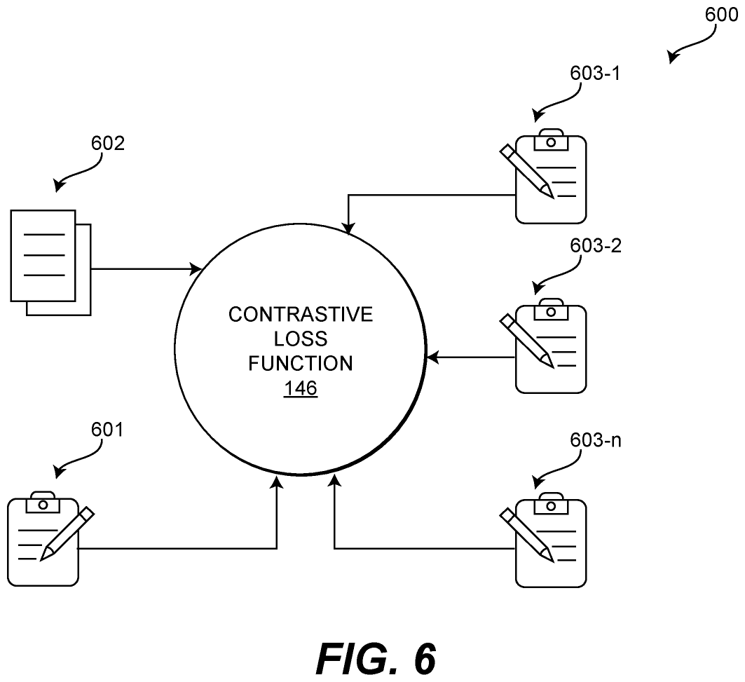

FIG. 4 is a flowchart of an example process 400 for training an AI model for validating a classification code that is assigned to a data object. FIG. 5 is a diagram of an example process 500 for training an AI model for validating a classification code that is assigned to a data object. FIG. 6 is a diagram of an example process 600 for training a contrastive loss function.

Process 400 can be performed by the validation system 130. As shown in FIG. 4 at block 410, the process 400 can include receiving training data including data objects, classification codes assigned to the data objects, and labels indicating whether the classification codes assigned to the data objects are correct. As further shown in FIG. 4 at block 420, the process 400 can include training an AI model to validate a classification code assigned to a data object using the training data. As further shown in FIG. 4 at block 430, the process 400 can include deploying the AI model.

The training data can include a “positive data object.” A positive data object can refer to a data object that is associated with an assigned classification code that has been validated as being correct. Further, the training data can include a “negative data object.” A negative data object can refer to a data object that is associated with an assigned classification code that has been validated as being incorrect. For example, as shown in FIG. 5, the BERT model 142 can receive a positive data object 501 and a negative data object 502. The BERT model 142 can be pre-trained. In this case, the BERT model 142 can be re-trained on unlabeled text of the positive data object 501 and the negative data object 502 to generate a semantic representation of the positive data object 501 and the negative data object 502. The FFN 144 can act as a projection layer, and be trained to map initialized representations of the positive data object 501 and the negative data object 502 and classification codes. The contrastive loss function 146 can be trained using the classification code 503 to learn and identify the representation of the positive data object 501 and the representation of the initialized classification code.

For instance, given an entity's history of admission sequences

$${E}_{1 : t}^{p} = \{ {x}_{1}^{p} , {x}_{2}^{p} , … , {x}_{t - 1}^{p} , {x}_{t}^{p} \} ,$$

the classification code model 120 can assign classification codes to each of the admission sequences to a particular entity

$$p {H}_{1 : t}^{p} = \{ {h}_{1}^{p} , {h}_{2}^{p} , … , {h}_{t - 1}^{p} , {h}_{t}^{p} \} ,$$

where hₜ^{p} is the assigned classification code to entity p for the t-th visit. The predicted classification codes can be validated and labeled

$${L}_{1 : t}^{p} = \{ {l}_{1}^{p} , {l}_{2}^{p} , … , {l}_{t - 1}^{p} , {l}_{t}^{p} \} .$$

$${l}_{i}^{p} = \{ \begin{matrix}1 , & if assigned {h}_{i}^{p} is correct \\ 0 , & if assigned {h}_{i}^{p} is incorrect\end{matrix}$$

For every data object

$${x}_{i}^{p}$$

of entity p's i^{th} visit, the valuation system 130 can sample negative data objects

$$\{ {\hat{x}}_{1}^{p} , {\hat{x}}_{2}^{p} , … , {\hat{x}}_{i}^{p} \}$$

such that:

$$\{ {l}_{1}^{p} , {l}_{2}^{p} , … , {l}_{j}^{p} \} = \{ 0 \}$$

Given an entity's history of admission sequences

$${E}_{1 : t}^{p} = \{ {x}_{1}^{p} , {x}_{2}^{p} , … , {x}_{t - 1}^{p} , {x}_{t}^{p} \} ,$$

the goal of training the AI model 140 is to validate whether the assigned classification code is correct or not yₜ={0,1} at time t, which can be regarded as a binary classification task.

The validation system 130 can use semi-supervised learning and contrastive supervised learning by leveraging label information. Normalized embeddings from the same class (e.g., between positive data objects and classification codes) are pulled closer together than embeddings from different clinical texts (e.g., negative data objects). The validation system 130 can use a single positive data object and multiple negative data objects per anchor. For example, as shown in FIG. 6, the contrastive loss function 146 can be trained using a positive data object 601, an anchor 602, and negative data objects 603-1 through 603-n. In this way, the AI model 140 can be trained using a larger number of the negative data objects than the positive data objects per anchor.

The validation system 130 can use a joint semi-supervised learning framework to learn the contextual representation of classification codes and data objects. Because classification codes are sets of ICD codes with no formal textual description, generating a representation of classification codes can be useful for other downstream tasks.

The validation system 130 can apply contrastive loss for validation or existence of classification codes for cross-domain assignment. Specifically, the validation system 130 can pre-train the BERT model 142 with data objects from a first domain and a second domain, then fine tune the model on data objects from the second domain with labels using contrastive loss function. Further, the validation system 130 can compute the gradient of the loss, and use the tuned BERT model 142 for assigning classification codes to data objects from the second domain using normalized cosine similarity.

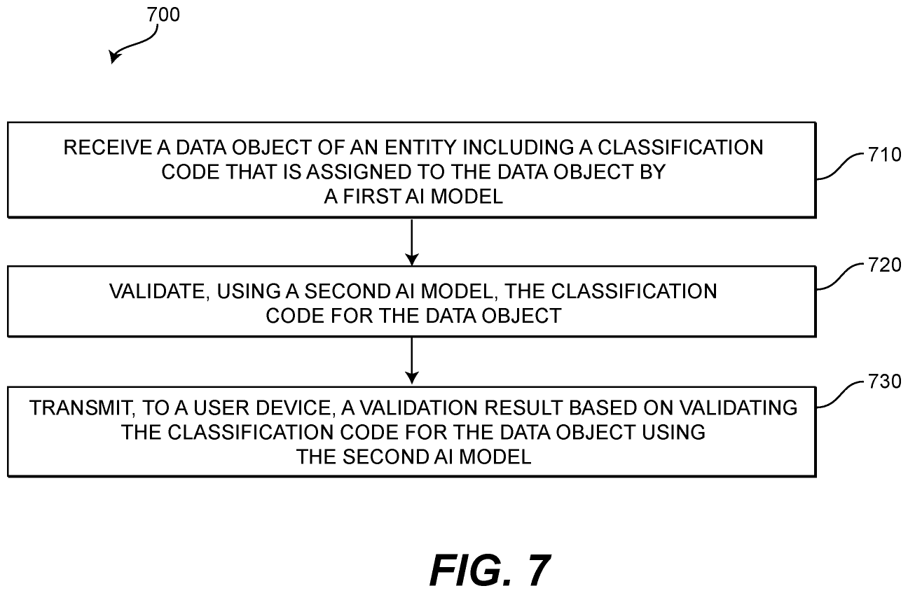

FIG. 7 is a flowchart of an example process 700 for validating a classification code that is assigned to a data object using an AI model. Process 700 can be performed by the validation system 130. As shown in FIG. 7 at block 710, the process 700 can include receiving a data object of a patient including a classification code that is assigned to the data object by a first AI model. As further shown in FIG. 7 at block 720, the process 700 can include validating, using a second AI model, the classification code for the data object assigned by the first AI model. As further shown in FIG. 7 at block 730, the process 700 can include transmitting, to a user device, a validation result based on validating the classification code for the data object using the second AI model.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the disclosure is not to be considered as limited by the foregoing description.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure furthermore relates to the following aspects.

Example 1. A computer-implemented method can include receiving, by one or more processors, a data object associated with an entity including a classification code that is assigned to the data object via a first artificial intelligence (AI) model; validating, by the one or more processors and using a second AI model, the classification code for the data object that is assigned to the data object via the first AI model, wherein the second AI model is trained using positive data objects that include assigned classification codes that are correct and negative data objects that include assigned classification codes that are incorrect; and transmitting, by the one or more processors and to a user device, a validation result based on validating the classification code for the data object using the second AI model

Example 2. The computer-implemented method of Example 1, wherein the second AI model includes a bidirectional encoder representations from transformers (BERT) model that is configured to generate a semantic representation of the data object.

Example 3. The computer-implemented method of Example 2, wherein the second AI model further includes a feed-forward network (FFN) that is configured to map the semantic representation and the classification code to a space where contrastive loss is applied.

Example 4. The computer-implemented method of Example 3, wherein the second AI model further includes a contrastive loss function configured to identify whether the classification code is correct.

Example 5. The computer-implemented method of any of Examples 1-4, wherein the classification code is a hierarchical condition category (HCC) code.

Example 6. The computer-implemented method of Example 5, wherein the data object is an electronic health record (EHR) and the entity is a patient.

Example 7. The computer-implemented method of any of Examples 1-6, wherein the second AI model is trained using a larger number of the negative data objects than the positive data objects per anchor.

Example 8. A device can include a memory configured to store instructions; and one or more processors configured to execute the instructions to perform operations comprising: receiving, by the one or more processors, a data object associated with an entity including a classification code that is assigned to the data object via a first artificial intelligence (AI) model; validating, by the one or more processors and using a second AI model, the classification code for the data object that is assigned to the data object via the first AI model, wherein the second AI model is trained using positive data objects that include assigned classification codes that are correct and negative data objects that include assigned classification codes that are incorrect; and transmitting, by the one or more processors and to a user device, a validation result based on validating the classification code for the data object using the second AI model.

Example 9. The device of Example 8, wherein the second AI model includes a bidirectional encoder representations from transformers (BERT) model that is configured to generate a semantic representation of the data object.

Example 10. The device of Example 9, wherein the second AI model further includes a feed-forward network (FFN) that is configured to map the semantic representation and the classification code to a space where contrastive loss is applied.

Example 11. The device of Example 10, wherein the second AI model further includes a contrastive loss function configured to identify whether the classification code is correct.

Example 12. The device of any of Examples 8-11, wherein the classification code is a hierarchical condition category (HCC) code.

Example 13. The device of example 12, wherein the data object is an electronic health record (EHR) and the entity is a patient.

Example 14. The device of any of Examples 8-13, wherein the second AI model is trained using a larger number of the negative data objects than the positive data objects per anchor.

Example 15. A non-transitory computer-readable medium can store instructions that, when executed by one or more processors, cause the one or more processors to perform operations comprising: receiving, by one or more processors, a data object associated with an entity including a classification code that is assigned to the data object via a first artificial intelligence (AI) model; validating, by the one or more processors and using a second AI model, the classification code for the data object that is assigned to the data object via the first AI model, wherein the second AI model is trained using positive data objects that include assigned classification codes that are correct and negative data objects that include assigned classification codes that are incorrect; and transmitting, by the one or more processors and to a user device, a validation result based on validating the classification code for the data object using the second AI model.

Example 16. The non-transitory computer-readable medium of Example 15, wherein the second AI model includes a bidirectional encoder representations from transformers (BERT) model that is configured to generate a semantic representation of the data object.

Example 17. The non-transitory computer-readable medium of Example 16, wherein the second AI model further includes a feed-forward network (FFN) that is configured to map the semantic representation and the classification code to a space where contrastive loss is applied.

Example 18. The non-transitory computer-readable medium of Example 17, wherein the second AI model further includes a contrastive loss function configured to identify whether the classification code is correct.

Example 19. The non-transitory computer-readable medium of any of Examples 15-18, wherein the classification code is a hierarchical condition category (HCC) code.

Example 20. The non-transitory computer-readable medium of any of Examples 15-19, wherein the data object is an electronic health record (EHR) and the entity is a patient.

We claim:

1. A computer-implemented method comprising:
receiving, by one or more processors, a data object associated with an entity including a classification code that is assigned to the data object via a first artificial intelligence (AI) model;
validating, by the one or more processors and using a second AI model, the classification code for the data object that is assigned to the data object via the first AI model, wherein the second AI model is trained using positive data objects that include assigned classification codes that are correct and negative data objects that include assigned classification codes that are incorrect; and
transmitting, by the one or more processors and to a user device, a validation result based on validating the classification code for the data object using the second AI model.

2. The computer-implemented method of claim 1, wherein the second AI model includes a bidirectional encoder representations from transformers (BERT) model that is configured to generate a semantic representation of the data object.

3. The computer-implemented method of claim 2, wherein the second AI model further includes a feed-forward network (FFN) that is configured to map the semantic representation and the classification code to a space where contrastive loss is applied.

4. The computer-implemented method of claim 3, wherein the second AI model further includes a contrastive loss function configured to identify whether the classification code is correct.

5. The computer-implemented method of claim 1, wherein the classification code is a hierarchical condition category (HCC) code.

6. The computer-implemented method of claim 5, wherein the data object is an electronic health record (EHR) and the entity is a patient.

7. The computer-implemented method of claim 1, wherein the second AI model is trained using a larger number of the negative data objects than the positive data objects per anchor.

8. A device comprising:
a memory configured to store instructions; and
one or more processors configured to execute the instructions to perform operations comprising:
receiving, by the one or more processors, a data object associated with an entity including a classification code that is assigned to the data object via a first artificial intelligence (AI) model;
validating, by the one or more processors and using a second AI model, the classification code for the data object that is assigned to the data object via the first AI model, wherein the second AI model is trained using positive data objects that include assigned classification codes that are correct and negative data objects that include assigned classification codes that are incorrect; and
transmitting, by the one or more processors and to a user device, a validation result based on validating the classification code for the data object using the second AI model.

9. The device of claim 8, wherein the second AI model includes a bidirectional encoder representations from transformers (BERT) model that is configured to generate a semantic representation of the data object.

10. The device of claim 9, wherein the second AI model further includes a feed-forward network (FFN) that is configured to map the semantic representation and the classification code to a space where contrastive loss is applied.

11. The device of claim 10, wherein the second AI model further includes a contrastive loss function configured to identify whether the classification code is correct.

12. The device of claim 8, wherein the classification code is a hierarchical condition category (HCC) code.

13. The device of claim 12, wherein the data object is an electronic health record (EHR) and the entity is a patient.

14. The device of claim 8, wherein the second AI model is trained using a larger number of the negative data objects than the positive data objects per anchor.

15. A non-transitory computer-readable medium storing instructions that, when executed by one or more processors, cause the one or more processors to perform operations comprising:
receiving, by one or more processors, a data object associated with an entity including a classification code that is assigned to the data object via a first artificial intelligence (AI) model;
validating, by the one or more processors and using a second AI model, the classification code for the data object that is assigned to the data object via the first AI model, wherein the second AI model is trained using positive data objects that include assigned classification codes that are correct and negative data objects that include assigned classification codes that are incorrect; and
transmitting, by the one or more processors and to a user device, a validation result based on validating the classification code for the data object using the second AI model.

16. The non-transitory computer-readable medium of claim 15, wherein the second AI model includes a bidirectional encoder representations from transformers (BERT) model that is configured to generate a semantic representation of the data object.

17. The non-transitory computer-readable medium of claim 16, wherein the second AI model further includes a feed-forward network (FFN) that is configured to map the semantic representation and the classification code to a space where contrastive loss is applied.

18. The non-transitory computer-readable medium of claim 17, wherein the second AI model further includes a contrastive loss function configured to identify whether the classification code is correct.

19. The non-transitory computer-readable medium of claim 15, wherein the classification code is a hierarchical condition category (HCC) code.

20. The non-transitory computer-readable medium of claim 15, wherein the data object is an electronic health record (EHR) and the entity is a patient.
